(19) United States
(12) Patent Application Publication
(10) Pub. No.: US 2025/0177272 A1
(43) Pub. Date: Jun. 5, 2025

(54) WATER-IN-OIL SUNSCREEN COSMETIC

(71) Applicant: SHISEIDO COMPANY, LTD., Chuo-ku, Tokyo (JP)

(72) Inventors: Rei NAKAMURA, Tokyo (JP); Yukiko SATO, Tokyo (JP)

(73) Assignee: SHISEIDO COMPANY, LTD., Chuo-ku, Tokyo (JP)

(21) Appl. No.: 18/843,912

(22) PCT Filed: Apr. 26, 2023
(86) PCT No.: PCT/JP2023/016534

§ 371 Date: Sep. 4, 2024

(30) Foreign Application Priority Data
May 10, 2022 (JP) JP2022-077324

(51) Int. Cl. A61K 8/40; A61K 8/06; A61K 8/25; A61K 8/34; A61Q 17/04

(52) U.S. Cl. CPC A61K 8/40; A61K 8/064; A61K 8/25; A61K 8/345; A61Q 17/04

(57) ABSTRACT

[Problem] An objective of the present invention is to provide a low-viscosity water-in-oil sunscreen cosmetic that suppresses powdery squeakiness in a coating film and unnatural whiteness of finish, and that has strong ultraviolet protection effects. [Solution] The water-in-oil sunscreen cosmetic of the present invention contains (A) an ultraviolet absorbing agent containing octocrylene, and (B) a dihydric alcohol having an IOB of 2.0 or lower, wherein a blended percentage of ethylhexyl methoxycinnamate relative to a total blended amount of component (A) is 15% or less, a blended amount of the ultraviolet scattering agent is 5% by mass or less relative to the overall amount of the cosmetic, and the water-in-oil sunscreen cosmetic has a viscosity of 1000 mPa·s or lower.

### TECHNICAL FIELD

The present invention relates to a water-in-oil sunscreen cosmetic that suppresses powderiness in a coating film and unnatural whiteness of finish, and which has strong ultraviolet protection effects.

### BACKGROUND ART

In order to protect skin from harm due to ultraviolet rays, ultraviolet protectants, such as ultraviolet absorbing agents and ultraviolet scattering agents, are blended into cosmetics. Ultraviolet absorbing agents have the advantages of being highly transparent, having good spreadability on the skin and not being easily broken up by perspiration. However, in some cases, they can cause an undesirable texture in use, such as oiliness or stickiness. On the other hand, ultraviolet scattering agents, which are inorganic powder components such as zinc oxide and titanium oxide, reduce the harm that ultraviolet rays cause to skin by scattering/reflecting the ultraviolet rays by physical mechanisms. Therefore, although they lack problems relating to oiliness and stickiness as is the case with ultraviolet absorbing agents, when cosmetics in which ultraviolet scattering agents have been blended are applied to skin, there are cases in which they cause unnatural whiteness and powdery squeakiness (a sensation of a powder scratching against or catching on the skin when a cosmetic is applied) to occur.

Thus, in order to realize high ultraviolet protection effects (high SPF) in sunscreen cosmetics, it is common to combine and blend ultraviolet absorbing agents and ultraviolet scattering agents in a well-balanced manner while utilizing the advantages of each.

Meanwhile, when a cosmetic that has been applied to skin is exposed to light or heat, the ultraviolet absorbing agents and other components contained in the cosmetic are degraded and the ultraviolet protection effects are reduced. Thus, in order to obtain high ultraviolet protection effects in a sunscreen cosmetic, technology for preventing decreases in the ultraviolet protection effects due to light and heat is considered to be necessary.

Patent Document 1 describes that, by blending 5% by mass or more of an amphiphilic substance and an ester oil with an IOB of 0.05-0.60 into a cosmetic containing 4-tert-butyl-4′-methoxydibenzoylmethane and ethylhexyl methoxycinnamate, photodegradation of the ultraviolet protection performance of the 4-tert-butyl-4′-methoxydibenzoylmethane and the ethylhexyl methoxycinnamate is suppressed, and a sunscreen cosmetic having high ultraviolet protection effects is obtained.

Additionally, Patent Document 2 reports that a humectant or an amphiphilic substance having an IOB value of 5.0 or lower has higher effects of enhancing the ultraviolet protection performance of an ultraviolet protectant after heat has been applied to a coating film than immediately after a cosmetic has been applied.

Thus, in a sunscreen cosmetic, there are cases in which a technology is necessary for obtaining high ultraviolet protection effects in accordance with an ultraviolet protectant that has been selected in order to realize a format and a feel in use that are sought.

### RELATED ART

### Patent Documents

Patent Document 1: WO 2017/057676
Patent Document 2: WO 2020/032246

### SUMMARY OF THE INVENTION

### Problem to be Solved by the Invention

In the course of research by the inventors, etc., there was a problem in that, in order to realize a format that can be easily applied, such as a spray type, a low-viscosity sunscreen cosmetic is required, yet, if the viscosity is low, powdery squeakiness, mainly caused by ultraviolet scattering agents, tends to be more easily felt. Additionally, when the blended amount of ultraviolet scattering agents is reduced in order to prevent powdery squeakiness and unnatural whiteness, high ultraviolet protection effects are difficult to realize.

Thus, an objective of the present invention is to provide a low-viscosity water-in-oil sunscreen cosmetic that suppresses powdery squeakiness in a coating film and unnatural whiteness of finish, and that has strong ultraviolet protection effects.

### Means for Solving the Problem

The present inventors performed diligent research towards solving the above-mentioned problem, as a result of which they discovered that, in a cosmetic containing octocrylene, a dihydric alcohol having an IOB of 2.0 or lower, due to heat being applied to a coating film, amplifies the ultraviolet protection performance of an ultraviolet protectant.

That is, the present invention provides a water-in-oil sunscreen cosmetic comprising:
(A) an ultraviolet absorbing agent containing octocrylene, and
(B) a dihydric alcohol having an IOB of 2.0 or lower,
   wherein a blended percentage of ethylhexyl methoxycinnamate relative to a total blended amount of component (A) is 15% or less,
   a blended amount of the ultraviolet scattering agent is 5% by mass or less relative to the overall amount of the cosmetic, and
   the water-in-oil sunscreen cosmetic has a viscosity of 1000 mPa·s or lower.

### Effects of the Invention

Due to the cosmetic of the present invention having the above-mentioned features, it is possible to obtain a sunscreen cosmetic having strong ultraviolet protection effects despite the blended amounts of ethylhexyl methoxycinnamate and an ultraviolet scattering agent being low. Additionally, in the sunscreen cosmetic according to the present invention, the blended amount of the ultraviolet scattering agent can be reduced. Therefore, it has the advantage that, despite being low in viscosity, there is no powdery squeakiness and unnatural whiteness does not occur after being applied.

### MODES FOR CARRYING OUT THE INVENTION

The cosmetic according to the present invention is characterized by comprising (A) an ultraviolet absorbing agent containing octocrylene, and (B) a dihydric alcohol having an IOB of 2.0 or lower. Hereinafter, the respective components constituting the cosmetic of the present application will be explained in detail.

### <(A) Ultraviolet Absorbing Agent>

The (A) ultraviolet absorbing agent (hereinafter sometimes referred to simply as “component (A)”) blended into the cosmetic according to the present invention refers to those that are normally blended into cosmetics for the purposes of absorbing energy by chemical mechanisms and converting the energy to heat or the like in order to prevent the ultraviolet rays from affecting skin cells, and essentially includes octocrylene, which is a β,β-diphenyl acrylate derivative.

Examples of ultraviolet absorbing agents other than octocrylene include benzoic acid derivatives, salicylic acid derivatives, cinnamic acid derivatives, dibenzoyl methane derivatives, benzophenone derivatives, benzylidene camphor derivatives, phenylbenzimidazole derivatives, triazine derivatives, phenylbenzotriazole derivatives, anthranil derivatives, imidazoline derivatives, benzalmalonate derivatives, 4,4-diaryl butadiene derivatives and the like. Hereinafter, specific examples and product names will be listed, but there is no limitation thereto.

Examples of benzoic acid derivatives include ethyl para-aminobenzoate (PABA), ethyl-dihydroxypropyl PABA, ethylhexyl-dimethyl PABA (e.g., “Escalol 507”; ISP), glyceryl PABA, PEG-25-PABA (e.g., “Uvinul P25”; BASF), diethylamino hydroxybenzoyl hexyl benzoate (e.g., “Uvinul A Plus”; BASF) and the like.

Examples of salicylic acid derivatives include homosalate (“Eusolex HMS”; Rona/EM Industries), ethylhexyl salicylate or ethylhexyl salicylic acid (e.g., “Neo Heliopan OS”; Haarmann & Reimer), dipropylene glycol salicylate (e.g., “Dipsal”; Scher), TEA salicylate (e.g., “Neo Heliopan TS”; Haarmann & Reimer) and the like.

Examples of cinnamic acid derivatives include octyl methoxycinnamate or ethylhexyl methoxycinnamate (e.g., “Parsol MCX”; Hoffmann-La Roche), isopropyl methoxycinnamate, isoamyl methoxycinnamate (e.g., “Neo Heliopan E1000”; Haarmaan & Reimer), cinnoxate, DEA methoxycinnamate, diisopropyl methyl cinnamate, glyceryl ethylhexanoate dimethoxycinnamate, di-(2-ethylhexyl)-4′-methoxybenzalmalonate and the like.

Examples of dibenzoyl methane derivatives include 4-tert-butyl-4′-methoxy dibenzoyl methane (e.g., “Parsol 1789”; DSM Nutrition Japan) and the like.

Examples of benzophenone derivatives include benzophenone-1 (e.g., “Uvinul 400”; BASF), benzophenone-2 (e.g., “Uvinul D50”; BASF), benzophenone-3 or oxybenzone (e.g., “Uvinul M40”; BASF), benzophenone-4 (e.g., “Uvinul MS40”; BASF), benzophenone-5, benzophenone-6 (e.g., “Helisorb 11”; Norquay), benzophenone-8 (e.g., “Spectra-Sorb UV-24”; American Cyanamid), benzophenone-9 (e.g., “Uvinul DS-49”; BASF), benzophenone-12 and the like.

Examples of benzylidene camphor derivatives include 3-benzylidene camphor (e.g., “Mexoryl SD”; Chimex), 4-methylbenzylidene camphor, benzylidene camphor sulfonic acid (e.g., “Mexoryl SL”; Chimex), camphor benzalkonium methosulfate (e.g., “Mexoryl SO”; Chimex), terephthalylidene dicamphor sulfonic acid (e.g., “Mexoryl SX”; Chimex), polyacrylamide methylbenzylidene camphor (e.g., “Mexoryl SW”; Chimex) and the like.

Examples of phenylbenzimidazole derivatives include phenylbenzimidazole sulfonic acid (e.g., “Eusolex 232”; Merck), disodium phenyldibenzimidazole tetrasulfonate (e.g., “Neo Heliopan AP”; Haarmann & Reimer) and the like.

Examples of triazine derivatives include bis-ethylhexyloxyphenol methoxyphenyl triazine (e.g., “Tinosorb S”; Ciba Specialty Chemicals), ethylhexyl triazone (e.g., “Uvinul T150”; BASF), diethylhexyl butamido triazone (e.g., “Uvasorb HEB”; Sigma 3V), 2,4,6-tris(diisobutyl-4′-aminobenzalmalonate)-s-triazine, 2,4,6-tris [4-(2-ethylhexyloxycarbonyl) anilino]-1,3,5-triazine and the like.

Examples of phenylbenzotriazole derivatives include drometrizole trisiloxane (e.g., “Silatrizole”; Rhodia Chimie), methylene bis(benzotriazolyl tetramethylbutyl phenol) (e.g., “Tinosorb M” (Ciba Specialty Chemicals)) and the like.

Examples of anthranil derivatives include menthyl anthranilate (e.g., “Neo Heliopan MA”; Haarmann & Reimer) and the like.

Examples of imidazoline derivatives include ethylhexyl dimethoxybenzylidene dioxoimidazoline propionate and the like.

Examples of benzalmalonate derivatives include polyorganosiloxanes having benzalmalonate functional groups (e.g., Polysilicone-15; “Parsol SLX”; DSM Nutrition Japan) and the like.

Examples of 4,4-diarylbutadiene derivatives include 1,1-dicarboxy (2,2′-dimethylpropyl)-4,4-diphenylbutadiene and the like.

As component (A) used in the present invention, just octocrylene may be used alone, or octocrylene and one or more other types of ultraviolet absorbing agents may be combined and used.

The blended amount of component (A) is 3% to 40% by mass, preferably 5% to 30% by mass relative to the overall amount of the cosmetic. If the blended amount of component (A) is less than 3% by mass, sufficient ultraviolet protection effects are difficult to obtain, and even if more than 40% by mass is blended, an increase in ultraviolet protection effects that is commensurate with the blended amount cannot be expected, and the conditions become undesirable due to the stability and the texture in use becoming worse, etc.

In general, ethylhexyl methoxycinnamate is often blended into sunscreen cosmetics in order to achieve a high SPF. However, in the sunscreen cosmetic according to the present invention, since ethylhexyl methoxycinnamate tends to hinder increases in ultraviolet protection performance due to heating when component (A) and component (B) in the present invention are combined, in order to draw out the ultraviolet protection performance improvement effects due to component (B) to be described below to the maximum extent, the blended percentage of ethylhexyl methoxycinnamate relative to the total blended amount of the (A) ultraviolet absorbing agent, in terms of the percentage by mass, is 15% or less, and preferably 10% or less. Additionally, the cosmetic according to the present invention does not need to include ethylhexyl methoxycinnamate. Therefore, the embodiments of the cosmetic according to the present invention include embodiments not containing ethylhexyl methoxycinnamate.

The sunscreen cosmetic according to the present invention can obtain high ultraviolet protection effects by blending only the aforementioned component (A). Thus, it is possible to reduce the blended amount of, or to not blend, an ultraviolet scattering agent such as zinc oxide, titanium oxide or the like. In this case, ultraviolet scattering agents refer to powders of zinc oxide, titanium oxide or the like, having a refractive index of 1.5 or higher, and in the form of fine particles having an average particle size of 0.1 μm or smaller. The embodiments of the cosmetic according to the present invention include embodiments in which the blended amount of the ultraviolet scattering agent is 5% by mass or less, 3% by mass or less, or 1% by mass or less relative to the overall amount of the cosmetic, as well as embodiments not containing an ultraviolet scattering agent.

It was discovered that, in the cosmetic according to the present invention, although the ultraviolet protection performance of the cosmetic itself can be increased by blending an ultraviolet scattering agent, it is difficult to obtain enhanced ultraviolet protection effects due to heat. Thus, in the cosmetic according to the present invention, ultraviolet protection performance improvement effects due to heat are sufficiently obtained by decreasing the blended amount of the ultraviolet scattering agent or by not blending an ultraviolet scattering agent. Additionally, in the cosmetic according to the present invention, a sunscreen cosmetic that does not cause unnatural whiteness and does not have powdery squeakiness when applied to the skin can be obtained by decreasing the blended amount of the ultraviolet scattering agent or by not blending an ultraviolet scattering agent.

### <(B) Dihydric Alcohol>

The (B) dihydric alcohol (hereinafter sometimes referred to simply as “component (B)”) blended into the cosmetic according to the present invention refers to those that are normally used in cosmetics, having an IOB of 2.0 or lower. Specific examples include pentylene glycol (IOB=2.0), dipropylene glycol (IOB=1.84), hexylene glycol (IOB=1.8), ethoxydiglycol (IOB=1.2) and the like, among which it is preferable to use dipropylene glycol.

The blended amount of component (B) is 3% to 10% by mass, preferably 4% to 7% by mass relative to the overall amount of the cosmetic. If the blended amount of component (B) is less than 3% by mass, sufficient ultraviolet protection performance improvement effects are difficult to obtain, and if 10% by mass is exceeded, there are cases in which the stability is reduced and the feel in use becomes worse, such as becoming sticky.

The sunscreen cosmetic according to the present invention has a viscosity of 1,000 mPa·s or lower. The lower limit value of the viscosity should preferably be 200 mPa·s or higher. Thus, a favorable viscosity range is 200 to 1,000 mPa·s. The viscosity in the present specification is the value measured by a B-type viscometer at 30° C.

In the sunscreen cosmetic according to the present invention, a (C) hydrophobized silica may be further blended in addition to the aforementioned components (A) and (B) from the aspect of further increasing the ultraviolet protection performance that the cosmetic itself has. In the present invention, the ultraviolet protection performance that the cosmetic has before heat is applied can be increased by blending a combination of component (B) and component (C) with the aforementioned component (A).

The (C) hydrophobized silica (hereinafter sometimes referred to simply as “component (C)”) blended into the cosmetic according to the present invention refers to a silica (silicic anhydride) that is normally used in the field of cosmetics, having a particle surface that is hydrophobic, having an average particle size of 2 to 8 μm, a specific surface area of 200 to 350 m²/g, and an oil absorption rate of 100 ml/100 g or higher, preferably 100 to 200 ml/100 g. In particular, a porous spherical silica is preferred.

The particle surface hydrophobization method of the (C) hydrophobized silica is not particularly limited. However, from the aspect of obtaining high ultraviolet protection performance, it is preferable to use a silicone such as methyl hydrogen polysiloxane or dimethyl polysiloxane as the hydrophobization treatment agent. Such hydrophobized silicas include, for example, SA-SB-150, SA-SB-300 (manufactured by Miyoshi Kasei), etc.

Regarding the blended amount of component (C), since it is an optional blended component in the cosmetic according to the present invention, it does not always need to be blended. However, if blended, it should be blended in an amount allowing the effects of the blending to be observed, and within limits such that the blended amount does not become excessive and problems such as degradation of the feel in use are not observed. A suitable blended amount for component (C) in the cosmetic according to the present invention is preferably 1% to 10% by mass or 2% to 8% by mass relative to the overall amount of the cosmetic.

By blending a combination of the aforementioned (B) dihydric alcohol and the aforementioned (C) hydrophobized silica in the cosmetic according to the present invention, higher ultraviolet protection effects than those obtained by blending the (C) hydrophobized silica alone can be obtained.

Aside from the aforementioned components (A) to (C), components that are normally used in cosmetics may be blended into the cosmetic according to the present invention within a range not compromising the effects of the present invention. For example, surfactants, oils, powder components, pH adjusters, chelators, preservatives, antioxidants, medicinal agents, alcohols, colorants, pigments and the like can be appropriately blended as needed.

The cosmetic according to the present invention can be provided not only as a sunscreen cosmetic, but also as a skin-care cosmetic such as a lotion, as a makeup base, as a hair cosmetic (including various types of hair products such as hair sprays or hair treatments for protecting the hair or the scalp from ultraviolet rays) or the like provided with sunscreen effects.

Additionally, although the form of the container of the present invention is not limited, it is suited to being contained as a base liquid for an aerosol cosmetic. Aerosol cosmetics refer to those in which a pressure-resistant valved can is filled with a base liquid, which is a water-in-oil emulsion, and liquefied petroleum gas, dimethyl ether, nitrogen, oxygen, carbon dioxide, a chlorofluorocarbon or the like, which is a propellant. In accordance with the purpose of use, it may be formulated in the form of a spray (mist), a foam (bubbles) or the like. However, from the aspect of facilitating application to the skin and hair, a spray form is preferred.

The sunscreen cosmetic according to the present invention has the property wherein the ultraviolet protection effects (also referred to as the “ultraviolet protection performance”) of a coating film increase due to heat. In this case, “the ultraviolet protection effects increase due to heat” can be generally defined as indicated below.

A prescribed amount of a sample of the cosmetic is dripped onto a measurement plate, applied over a prescribed area and dried to form a coating film. The absorbance of the coating film is measured by an spectrophotometer or the like over a range of 280 to 400 nm, and an absorbance integral value of the coating film is determined with reference to the absorbance of an uncoated measurement plate.

Next, the measurement plate on which the coating film has been formed is heated under prescribed conditions, and after returning to ambient temperature, the absorbance of the coating film is measured and the absorbance integral value is similarly determined.

The absorbance integral value change rate (absorbance change rate) before and after the heat treatment is calculated in accordance with the following equation.

Absorbance change rate (%)=(Absorbance integral value after heat treatment)/(absorbance integral value before heat treatment)×100

If the change rate exceeds 100%, then this is defined as an increase in (enhancement of) the ultraviolet protection effects.

Instead of the absorbance integral value, the absorbance at a single wavelength (in the ultraviolet range), a critical wavelength, or the SPF or the UVAPF (or “PFA”) calculated based on the measured absorbance may be used as an index.

The temperature to which the coating film is heated should preferably be within the range from 30° C. to 70° C., and may, for example, be a temperature of at least 32° C., at least 35° C., at least 37° C., or at least 40° C., and a temperature of at most 65° C., at most 60° C., at most 55° C., or at most 50° C. If the heating temperature exceeds 70° C., there are cases in which problems such as resin measurement plates melting occur.

In order to accurately evaluate the influence due to heat, the heating time should preferably be at least 1 minute, and should more preferably be at least 10 minutes. Although the upper limit of the heating time is not particularly limited, it should normally be at most 60 minutes, and preferably at most 30 minutes.

In the cosmetic according to the present invention, ultraviolet protection performance improvement effects are obtained by combining component (A) with component (B). Therefore, it is not essential to blend components other than component (B), for example, a humectant or an amphiphilic substance having an IOB value of 5.0 or lower, as reported in Patent Document 2. In the cosmetic according to the present invention, the blended amount of a humectant or an amphiphilic substance having an IOB value of 5.0 or lower may be less than 5% by mass, less than 3% by mass, less than 1% by mass, or less than 0.1% by mass relative to the overall amount of the cosmetic, or it may not be blended at all. The humectant or amphiphilic substance having an IOB value of 5.0 or lower other than component (B) in the present invention specifically refers to those indicated in (1) and (2) below.
(1) A polyoxyalkylene/polyoxyethylene copolymer dialkyl ether represented by the following formula (I):

R¹O-[(AO)ₘ(EO)ₙ]—R² (I)

(in formula (I), AO denotes an oxyalkylene group having 3 to 4 carbon atoms, EO denotes an oxyethylene group, R¹ and R² denote, independently of each other, a hydrogen atom or a hydrocarbon group having 1 to 4 carbon atoms, and 1≤m≤70 and 1≤n≤70).

Specific examples include PEG/PPG-9/2 dimethyl ether, PEG/PPG-17/4 dimethyl ether, PEG/PPG-14/7 dimethyl ether, PEG/PPG-11/9 dimethyl ether, PEG/PPG-55/28 dimethyl ether, PEG/PPG-36/41 dimethyl ether, PEG/PPG-6/3 dimethyl ether, PEG/PPG-8/4 dimethyl ether, PEG/PPG-6/11 dimethyl ether and PEG/PPG-14/27 dimethyl ether.
(2) A polyhydric alcohol selected from among the polyalkylene glycols represented by the following formula (II):

HO(RO)ₚH (II)

(in formula (II), RO denotes an oxyalkylene group having 2 to 4 carbon atoms, and p is 3 to 500), and butylene glycol, diglycerin, propanediol, erythritol, xylitol, methyl gluceth-10 and sorbitol.

Specific examples of polyalkylene glycols represented by formula (II) include polyethylene glycols 150 to 23000, such as polyethylene glycol 300, polyethylene glycol 400, polyethylene glycol 1500 and polyethylene glycol 20000.

The (B) dihydric alcohol in the present invention partially overlaps with, but is not completely the same as the amphiphilic substance described in Patent Document 1, and with the humectant and the amphiphilic substance described in Patent Document 2. For example, the dibutylene glycol (IOB=2.4) for which photodegradation suppression effects are obtained in Patent Document 1, and the PEG/PPG-9/2 dimethyl ether and the polyethylene glycol 300 with which ultraviolet protection performance improvement effects due to heat are obtained in Patent Document 2 were observed to have difficulty obtaining ultraviolet protection improvement effects due to heat in the present invention.

The embodiments of the cosmetic according to the present invention include embodiments not including the polyoxyalkylene/polyoxyethylene copolymer alkyl ether represented by formula (I), the polyalkylene glycol represented by formula (II), butylene glycol, diglycerin, propanediol, erythritol, xylitol, methyl gluceth-10 and sorbitol.

In the cosmetic according to the present invention, an ester oil having an IOB value of 0.3 or higher does not need to be blended for the purpose of obtaining ultraviolet protection performance improvement effects. Thus, the embodiments of the cosmetic according to the present invention include embodiments in which the blended amount of an ester oil having an IOB value of 0.3 or higher is less than 5% by mass, less than 3% by mass or less than 1% by mass relative to the overall amount of the cosmetic, and embodiments not containing an ester oil having an IOB value of 0.3 or higher. Specific examples of ester oils having an IOB value of 0.3 or higher include propylene glycol dicaprylate (IOB=0.32), di-2-ethylhexyl succinate (IOB=0.32), pentaerythritol tetra-2-ethylhexanoate (IOB=0.35), glyceryl tri-2-ethylhexanoate (IOB=0.36), pentaerythritol tetraoctanoate (IOB=0.35), diisopropyl sebacate (IOB=0.40), tripropylene glycol dineopentanoate (IOB=0.52) and the like.

In the cosmetic according to the present invention, an oil-phase thickener selected from among dextrin fatty acid esters, sucrose fatty acid esters, solid or semi-solid hydrocarbon oils, organically modified clay minerals and fatty acids, or salts thereof, does not need to be blended for the purpose of obtaining ultraviolet protection performance improvement effects. Thus, the embodiments of the cosmetic according to the present invention include embodiments in which the blended amount of an oil-phase thickener selected from among dextrin fatty acid esters, sucrose fatty acid esters, solid or semi-solid hydrocarbon oils, organically modified clay minerals, and fatty acids or salts thereof is less than 3% by mass, less than 1% by mass, less than 0.5% by mass or less than 0.1% by mass relative to the overall amount of the cosmetic, and embodiments not containing the oil-phase thickener.

Additionally, in the cosmetic according to the present invention, a dextrin fatty acid ester and/or an organically modified clay mineral does not need to be blended for the purpose of obtaining ultraviolet protection performance improvement effects. Thus, the embodiments of the cosmetic according to the present invention include embodiments in which the blended amount of a dextrin fatty acid ester and/or an organically modified clay mineral is less than 3% by mass, less than 1% by mass, less than 0.5% by mass or less than 0.1% by mass relative to the overall amount of the cosmetic, and embodiments not containing a dextrin fatty acid ester and an organically modified clay mineral. Specific examples of dextrin fatty acid esters include dextrin palmitate and the like, and specific examples of organically modified clay minerals include dimethyldistearyl ammonium hectorite (disteardimonium hectorite) and the like.

### EXAMPLES

Although the present invention will be explained in further detail by providing examples below, the present invention is not limited in any way thereby. Where not otherwise noted, the blended amounts are indicated in percentage by mass relative to the systems in which the relevant components are blended. Before specifically explaining each example, the evaluation methods that were employed will be explained.

### 1. Measurement of Ultraviolet Protection Performance of Cosmetic

Cosmetics (samples) according to each example were dripped, at a rate of 2 mg/cm², onto measurement plates (S plates) (5×5 cm V-groove PMMA plates, SPFMASTER-PA01), applied by finger for 60 seconds and dried for 15 minutes, after which the absorbances at 310 nm of the formed coating films were measured using a U-3500 self-recording spectrophotometer (manufactured by Hitachi). The higher the measurement value, the higher the ultraviolet protection performance.

### 2. Measurement of Ultraviolet Protection Performance Change Rate Due to Heat

Cosmetics (samples) according to each example were dripped, at a rate of 2 mg/cm², onto measurement plates (S plates) (5×5 cm V-groove PMMA plates, SPFMASTER-PA01), applied by finger for 60 seconds and dried for 15 minutes, after which the absorbances of the formed coating films were measured using a U-3500 self-recording spectrophotometer (manufactured by Hitachi). Using an uncoated plate as a control, the absorbances (Abs) were calculated by the equation below, and the measurement values were integrated from 280 nm to 400 nm to determine the absorbance at 310 nm.

Abs=−log(T/To)
T: transmittance of sample, To: transmittance of uncoated plate

Next, the plates having the coating films were placed in a constant temperature bath to perform heat treatments at 37° C. for 30 minutes, and the absorbances at 310 nm were determined in the same manner as above.

The absorbance change rate (thermal change rate) before and after heat irradiation was calculated for each sample in accordance with the following equation.

Thermal change rate (%)=[(absorbance at 310 nm after heat treatment)/(absorbance at 310 nm before heat treatment)]×100

In the cases in which the thermal change rate exceeded 100%, ultraviolet protection performance improvement effects are evaluated to have been obtained.

The water-in-oil sunscreen cosmetics having the compositions indicated in Table 1 and Table 2 below were prepared by conventional methods. Specifically, the powders were dispersed in the oil-based components that were mixed by using a homomixer, after which the well-mixed water-based components were added to obtain the cosmetics. The ultraviolet protection performances were measured for the prepared cosmetics in accordance with the aforementioned evaluation method. The results are also indicated in the tables.

TABLE 1

| Component name | Ex. 1 | Ex. 2 | Ex. 3 | Ex. 4 |
|---|---|---|---|---|
| Octocrylene | 5 | 5 | 5 | 5 |
| Homosalate | 10 | 10 | 10 | 10 |
| Ethylhexyl methoxycinnamate | — | — | — | — |
| Ethylhexyl salicylate | 5 | 5 | 5 | 5 |
| bis-Ethylhexyloxyphenol | 1.5 | 1.5 | 1.5 | 1.5 |
| methoxyphenyl triazine | | | | |
| Ethylhexyl triazone | 1.5 | 1.5 | 1.5 | 1.5 |
| Diethylamino hydroxybenzoyl | 3 | 3 | 3 | 3 |
| hexyl benzoate | | | | |
| Diisopropyl sebacate | 5 | 5 | 5 | — |
| Isododecane | 5 | 5 | 5 | 5 |
| Volatile dimethicone | 14.5 | 14.5 | 14.5 | 14.5 |
| Lauryl PEG-9 polydimethyl | 3.5 | 3.5 | 3.5 | 3.5 |
| polysiloxyethyl dimethicone | | | | |
| Sorbitan sesquiisostearate | 1 | 1 | 1 | 1 |
| Dimethyldistearyl ammonium | 0.5 | 0.5 | 0.5 | — |
| hectorite | | | | |
| Dextrin palmitate | 1 | 1 | 1 | 1 |
| Methyl polysiloxane-treated silica | — | — | 5 | — |
| Silica | — | 5 | — | — |
| Dipropylene glycol (IOB = 1.84) | 5 | 5 | 5 | 5 |
| 1,3-Butylene glycol (IOB = 2.4) | — | — | — | — |
| PEG/PPG-9/2 dimethyl ether | — | — | — | — |
| Polyethylene glycol 300 | — | — | — | — |
| Ethanol | 5 | 5 | 5 | 5 |
| Purified water | bal | bal | bal | bal |
| Total | 100 | 100 | 100 | 100 |
| Ultraviolet protection performance | 1.67 | 1.57 | 2.06 | 1.77 |
| Ultraviolet protection performance | 105% | 104% | 105% | 101% |
| change rate due to heat | | | | |

TABLE 2

| | Comp. | Comp. | Comp. | Comp. | Comp. |
|---|---|---|---|---|---|
| Component name | Ex. 1 | Ex. 2 | Ex. 3 | Ex. 4 | Ex. 5 |
| Octocrylene | — | 5 | 5 | 5 | 5 |
| Homosalate | 10 | 10 | 10 | 10 | 10 |
| Ethylhexyl methoxycinnamate | — | — | — | — | — |
| Ethylhexyl salicylate | 5 | 5 | 5 | 5 | 5 |
| bis-Ethylhexyloxyphenol | 1.5 | 1.5 | 1.5 | 1.5 | 1.5 |
| methoxyphenyl triazine | | | | | |
| Ethylhexyl triazone | 1.5 | 1.5 | 1.5 | 1.5 | 1.5 |
| Diethylamino hydroxybenzoyl hexyl | 3 | 3 | 3 | 3 | 3 |
| benzoate | | | | | |
| Diisopropyl sebacate | 5 | 5 | 5 | 5 | 5 |
| Isododecane | 5 | 5 | 5 | 5 | 5 |
| Volatile dimethicone | 14.5 | 14.5 | 14.5 | 14.5 | 14.5 |
| Lauryl PEG-9 polydimethyl | 3.5 | 3.5 | 3.5 | 3.5 | 3.5 |
| polysiloxyethyl dimethicone | | | | | |
| Sorbitan sesquiisostearate | 1 | 1 | 1 | 1 | 1 |
| Dimethyldistearyl ammonium hectorite | 0.5 | 0.5 | 0.5 | 0.5 | 0.5 |
| Dextrin palmitate | 1 | 1 | 1 | 1 | 1 |
| Methyl polysiloxane-treated silica | 5 | 5 | — | — | — |
| Silica | — | — | — | — | — |
| Dipropylene glycol (IOB = 1.84) | 5 | — | — | — | — |
| 1,3-Butylene glycol (IOB = 2.4) | — | — | 5 | — | — |
| PEG/PPG-9/2 dimethyl ether | — | — | — | 5 | — |
| Polyethylene glycol 300 | — | — | — | — | 5 |
| Ethanol | 5 | 5 | 5 | 5 | 5 |
| Purified water | bal | bal | bal | bal | bal |
| Total | 100 | 100 | 100 | 100 | 100 |
| Ultraviolet protection performance | 1.47 | 1.67 | 1.51 | 1.6 | 1.29 |
| Ultraviolet protection performance | 97% | 97% | 98% | 100% | 99% |
| change rate due to heat | | | | | |

As indicated in Table 2, the cosmetics of Comparative Example 1, in which octocrylene was not blended, and Comparative Example 2, in which component (B) of the present invention was not blended, both were not able to obtain ultraviolet protection performance improvement effects due to heat.

Additionally, ultraviolet protection performance improvement effects due to heat also were not able to be obtained in the cases in which component (B) in the present invention was replaced with dihydric alcohol (Comparative Example 3), which has an IOB of 2.4, with PEG/PPG-9/2 dimethyl ether (Comparative Example 4) or with polyethylene glycol 300 (Comparative Example 5).

In contrast therewith, as indicated in Table 1, high ultraviolet protection performance improvement effects due to heat were obtained with the cosmetics of Examples 1, 2 and 4, in which components (A) and (B) of the present invention were blended.

Furthermore, the cosmetic of Example 3, in which a (C) hydrophobized silica (methyl polysiloxane-treated silica) was blended in addition to components (A) and (B) in the present invention, had high ultraviolet protection performance despite containing the same total blended amount of ultraviolet absorbing agents as the cosmetics of Examples 1, 2 and 4. This indicates that the ultraviolet protection performance of the cosmetic was enhanced by combining component (B) and component (C) in the present invention.

Additionally, from a comparison between Example 2, in which silica is blended, and Example 3, in which (C) hydrophobized silica is blended, it was demonstrated that prominent ultraviolet protection improvement effects are obtained in the case in which the (C) hydrophobized silica is blended.

Next, water-in-oil sunscreen cosmetics in which the blended amounts of the ultraviolet scattering agent and ethylhexyl methoxycinnamate were changed so as to have the compositions indicated in Table 3 were prepared by a conventional method. Likewise, the ultraviolet protection performances of the prepared cosmetics were measured in accordance with the aforementioned evaluation method. The results are also indicated in the table.

TABLE 3

| | | | Comp. | | Comp. |
|---|---|---|---|---|---|
| Component name | Ex. 3 | Ex. 5 | Ex. 6 | Ex. 6 | Ex. 7 |
| Octocrylene | 5 | 5 | 5 | 5 | 5 |
| Homosalate | 10 | 10 | 10 | 10 | 10 |
| Ethylhexyl methoxycinnamate | — | — | — | 3 | 5 |
| Ethylhexyl salicylate | 5 | 5 | 5 | 5 | 5 |
| bis-Ethylhexyloxyphenol methoxyphenyl | 1.5 | 1.5 | 1.5 | 1.5 | 1.5 |
| triazine | | | | | |
| Ethylhexyl triazone | 1.5 | 1.5 | 1.5 | 1.5 | 1.5 |
| Diethylamino hydroxybenzoyl hexyl | 3 | 3 | 3 | 3 | 3 |
| benzoate | | | | | |
| Diisopropyl sebacate | 5 | 5 | 5 | 5 | 5 |
| Isododecane | 5 | 5 | 5 | 5 | 5 |
| Volatile dimethicone | 14.5 | 14.5 | 14.5 | 14.5 | 14.5 |
| Lauryl PEG-9 polydimethyl polysiloxyethyl | 3.5 | 3.5 | 3.5 | 3.5 | 3.5 |
| dimethicone | | | | | |
| Sorbitan sesquiisostearate | 1 | 1 | 1 | 1 | 1 |
| Dimethyldistearyl ammonium hectorite | 0.5 | 0.5 | 0.5 | 0.5 | 0.5 |
| Dextrin palmitate | 1 | 1 | 1 | 1 | 1 |
| Hydrophobically treated fine-particle zinc | — | 5 | 10 | — | — |
| oxide | | | | | |
| Methyl polysiloxane-treated silica | 5 | 5 | 5 | 5 | 5 |
| Dipropylene glycol | 5 | 5 | 5 | 5 | 5 |
| Ethanol | 5 | 5 | 5 | 5 | 5 |
| Purified water | bal | bal | bal | bal | bal |
| Total | 100 | 100 | 100 | 100 | 100 |
| Ultraviolet protection performance | 2.06 | 1.98 | 2.05 | 1.92 | 2.07 |
| Ultraviolet protection performance change | 105% | 101% | 99% | 101% | 100% |
| rate due to heat | | | | | |
| Blending percentage of ethylhexyl | — | — | — | 10.3% | 16.1% |
| methoxycinnamate relative to total amount | | | | | |
| of (A) ultraviolet absorbing agent | | | | | |

As indicated in Table 3, in the case in which the blended amount of the ultraviolet scattering agent was set to be 10% by mass relative to the overall amount of the cosmetic (Comparative Example 6), although the ultraviolet protection performance of the cosmetic itself increased, ultraviolet protection performance improvement effects due to heat were not able to be obtained. Additionally, with the cosmetic of Comparative Example 6, unnatural whiteness occurred after application, powdery squeakiness was felt, and the feel in use was poor.

Additionally, when the blending percentage of ethylhexyl methoxycinnamate relative to the total blended amount of component (A) in the present invention was set to be high, ultraviolet protection performance improvement effects due to heat became difficult to obtain (Comparative Example 7).

In contrast therewith, in the cosmetics of the present invention, as indicated by Examples 3, 5 and 6, higher ultraviolet protection performance was obtained and ultraviolet protection performance improvement effects due to heat were also able to be obtained when the blended amount of the ultraviolet scattering agent was low and the blending percentage of ethylhexyl methoxycinnamate relative to the total blended amount of the ultraviolet absorbing agent was low.

1. A water-in-oil sunscreen cosmetic comprising:
(A) an ultraviolet absorbing agent containing octocrylene, and
(B) a dihydric alcohol having an IOB of 2.0 or lower,
wherein a blended percentage of ethylhexyl methoxycinnamate relative to a total blended amount of component (A) is 15% or less,
a blended amount of the ultraviolet scattering agent is 5% by mass or less relative to the overall amount of the cosmetic, and
the water-in-oil sunscreen cosmetic has a viscosity of 1000 mPa·s or lower.

2. The water-in-oil sunscreen cosmetic according to claim 1, not containing ethylhexyl methoxycinnamate.

3. The water-in-oil sunscreen cosmetic according to claim 1, not containing an ultraviolet scattering agent.

4. The water-in-oil sunscreen cosmetic according to claim 1, wherein the component (B) is dipropylene glycol.

5. The water-in-oil sunscreen cosmetic according to claim 1, further comprising (C) hydrophobized silica.

6. The water-in-oil sunscreen cosmetic according to claim 1, which is an aerosol-type cosmetic.
